(19) United States
(12) Patent Application Publication
(10) Pub. No.: US 2025/0359988 A1
(43) Pub. Date: Nov. 27, 2025

(54) REPLACEMENT HEART VALVE IMPLANT SYSTEM

(71) Applicant: BOSTON SCIENTIFIC SCIMED, INC., MAPLE GROVE, MN (US)

(72) Inventors: Philip Andrew Litecky, Forest Lake, MN (US); Levi Joel Wolterstorff, Saint Paul, MN (US)

(73) Assignee: BOSTON SCIENTIFIC SCIMED, INC., MAPLE GROVE, MN (US)

(21) Appl. No.: 19/215,879

(22) Filed: May 22, 2025

Related U.S. Application Data
(60) Provisional application No. 63/651,556, filed on May 24, 2024.

(51) Int. Cl. A61F 2/24; A61F 2/95

(52) U.S. Cl. CPC A61F 2/2418; A61F 2/2436; A61F 2/95

(57) ABSTRACT

A replacement heart valve implant includes a framework and a plurality of valve leaflets. The framework includes interconnected struts, a plurality of upper crowns, and a plurality of lower crowns. The lower crowns include a bend line extending circumferentially through each lower crown and disposed upstream of upstreammost intersections of the struts and downstream of an upstreammost extent of the lower crowns. A replacement heart valve system includes the implant and an implant delivery system including a handle and an elongate shaft assembly, wherein a distal portion of the shaft assembly includes an implant holding portion. A method of loading the implant includes positioning the implant adjacent the holding portion, positioning the lower crowns surrounding a stent holder of the delivery system, collapsing the framework such that the lower crowns extend into a groove of the stent holder, and shifting the holding portion over the implant.

### CROSS-REFERENCE TO RELATED APPLICATION

This application is a continuation of U.S. Patent Application Ser. No. 63/651,556, filed May 24, 2024, entitled “REPLACEMENT HEART VALVE IMPLANT SYSTEM”, which is incorporated by reference herein in its entirety.

### TECHNICAL FIELD

The disclosure relates generally to medical devices and more particularly to a replacement heart valve implant and/or features for coupling a replacement heart valve implant to an implant delivery system.

### BACKGROUND

A wide variety of intracorporeal medical devices have been developed for medical use including replacement heart valve implants for repair or replacement of diseased heart valves. Of the known medical devices and methods, each has certain advantages and disadvantages. There is an ongoing need to provide alternative medical devices as well as alternative methods for manufacturing and using medical devices.

### SUMMARY

In one example, a replacement heart valve implant may comprise an expandable framework configured to shift between a radially collapsed configuration and a radially expanded configuration, and a plurality of valve leaflets secured to the expandable framework. The expandable framework may comprise a tubular wall including a plurality of interconnected struts, a plurality of upper crowns extending downstream from downstreammost intersections of the plurality of interconnected struts, and a plurality of lower crowns extending upstream from upstreammost intersections of the plurality of interconnected struts. The plurality of lower crowns may comprise a bend line extending circumferentially around a central longitudinal axis of the expandable framework through each lower crown, the bend line being disposed upstream of the upstreammost intersections of the plurality of interconnected struts and downstream of an upstreammost extent of the plurality of lower crowns.

In addition, or alternatively, to any example disclosed herein, the bend line defines a radially outermost extent of the plurality of lower crowns.

In addition, or alternatively, to any example disclosed herein, the upstreammost extent of the plurality of lower crowns is disposed radially inward of the radially outermost extent of the plurality of lower crowns.

In addition, or alternatively, to any example disclosed herein, an upstream portion of the plurality of lower crowns extending from the bend line upstream to the upstreammost extent of the plurality of lower crowns is oriented at an acute angle to the central longitudinal axis of the expandable framework.

In addition, or alternatively, to any example disclosed herein, the acute angle is greater than zero degrees and less than 45 degrees.

In addition, or alternatively, to any example disclosed herein, the acute angle is greater than 15 degrees and less than 30 degrees.

In addition, or alternatively, to any example disclosed herein, and in a second example, a replacement heart valve system may comprise a replacement heart valve implant, comprising an expandable framework configured to shift between a radially collapsed configuration and a radially expanded configuration, and a plurality of valve leaflets secured to the expandable framework, wherein the expandable framework comprises a tubular wall including a plurality of interconnected struts, a plurality of upper crowns extending downstream from downstreammost intersections of the plurality of interconnected struts, and a plurality of lower crowns extending upstream from upstreammost intersections of the plurality of interconnected struts, wherein the plurality of lower crowns comprises a bend line extending circumferentially around a central longitudinal axis of the expandable framework through each lower crown, the bend line being disposed upstream of the upstreammost intersections of the plurality of interconnected struts and downstream of an upstreammost extent of the plurality of lower crowns; and an implant delivery system comprising a handle and an elongate shaft assembly extending distally from the handle, wherein a distal portion of the elongate shaft assembly includes an implant holding portion configured to engage with the replacement heart valve implant in the radially collapsed configuration.

In addition, or alternatively, to any example disclosed herein, the implant holding portion comprises a proximal sheath configured to cover a proximal portion of the replacement heart valve implant in the radially collapsed configuration, and a distal sheath configured to cover a distal portion of the replacement heart valve implant in the radially collapsed configuration.

In addition, or alternatively, to any example disclosed herein, the implant holding portion comprises a stent holder having a groove extending circumferentially in an outer surface of the stent holder, the groove being configured to receive the plurality of lower crowns in the radially collapsed configuration.

In addition, or alternatively, to any example disclosed herein, the groove is configured to receive every lower crown of the plurality of lower crowns in the radially collapsed configuration.

In addition, or alternatively, to any example disclosed herein, the groove comprises an angled surface configured to matingly engage with an upstream portion of the plurality of lower crowns extending from the bend line upstream to the upstreammost extent of the plurality of lower crowns.

In addition, or alternatively, to any example disclosed herein, the groove comprises a first shoulder configured to engage the plurality of lower crowns at the bend line.

In addition, or alternatively, to any example disclosed herein, the stent holder is formed from a conformable material configured to extend between the plurality of lower crowns in the radially collapsed configuration.

In addition, or alternatively, to any example disclosed herein, the groove is formed by crimping the plurality of lower crowns onto the stent holder.

In addition, or alternatively, to any example disclosed herein, and in a third example, a method of loading a replacement heart valve implant into a replacement heart valve system may comprise: positioning a replacement heart valve implant adjacent an implant holding portion of an implant delivery system, the replacement heart valve implant comprising an expandable framework configured to shift between a radially collapsed configuration and a radially expanded configuration, and a plurality of valve leaflets secured to the expandable framework; wherein the expandable framework comprises a tubular wall including a plurality of interconnected struts, a plurality of upper crowns extending downstream from downstreammost intersections of the plurality of interconnected struts, and a plurality of lower crowns extending upstream from upstreammost intersections of the plurality of interconnected struts; wherein the plurality of lower crowns comprises a bend line extending circumferentially around a central longitudinal axis of the expandable framework through each lower crown, the bend line being disposed upstream of the upstreammost intersections of the plurality of interconnected struts and downstream of an upstreammost extent of the plurality of lower crowns; positioning the plurality of lower crowns surrounding a stent holder of the implant delivery system; radially collapsing the expandable framework to the radially collapsed configuration such that the plurality of lower crowns extends into a groove extending circumferentially in an outer surface of the stent holder; and shifting the implant holding portion of the implant delivery system over the replacement heart valve implant to a closed configuration.

In addition, or alternatively, to any example disclosed herein, an upstream portion of the plurality of lower crowns extending from the bend line upstream to the upstreammost extent of the plurality of lower crowns is oriented at an acute angle to the central longitudinal axis of the expandable framework.

In addition, or alternatively, to any example disclosed herein, the upstream portion of the plurality of lower crowns abuts an angled surface of the groove in the radially collapsed configuration.

In addition, or alternatively, to any example disclosed herein, the upstream portion of the plurality of lower crowns extends radially inward from the bend line prior to radially collapsing the expandable framework.

In addition, or alternatively, to any example disclosed herein, radially collapsing the expandable framework to the radially collapsed configuration bends the plurality of lower crowns radially inward at the bend line.

In addition, or alternatively, to any example disclosed herein, radially collapsing the expandable framework to the radially collapsed configuration forms the groove in the outer surface of the stent holder.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of a replacement heart valve implant;

FIG. 2 is a flat pattern drawing illustrating selected aspects of a replacement heart valve implant according to the disclosure;

FIG. 3 illustrates selected aspects of an implant delivery system for delivering a replacement heart valve implant;

FIG. 4 illustrates selected aspects of a portion of the implant delivery system of FIG. 3 according to the disclosure;

FIG. 5 illustrates selected aspects of a stent holder of the implant delivery system of FIGS. 3-4 engaging a replacement heart valve implant;

FIG. 6 illustrates selected aspects of a portion of the implant delivery system of FIG. 3 according to the disclosure;

FIG. 7 illustrates selected aspects of a stent holder of the implant delivery system of FIG. 6 prior to engaging a replacement heart valve implant;

FIG. 8 illustrates selected aspects of the stent holder of the implant delivery system of FIGS. 6-7 engaging a replacement heart valve implant; and

FIG. 9 is a partial cross-sectional view of FIG. 8 illustrating selected aspects of the stent holder of the implant delivery system engaging a replacement heart valve implant. While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

For the purpose of this disclosure, the discussion herein is directed toward use in treating a native heart valve such as the aortic valve and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure.

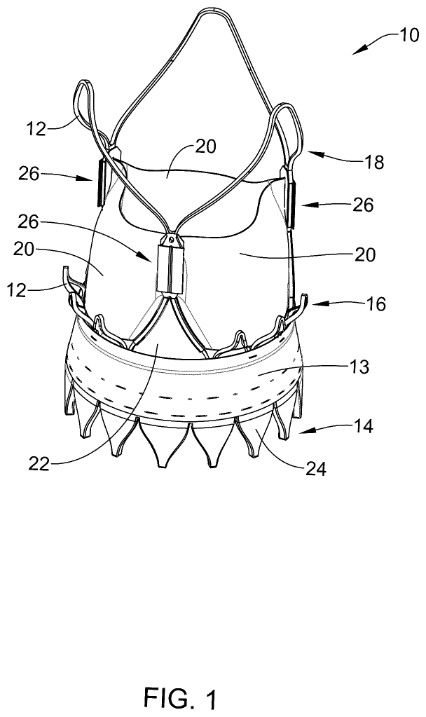

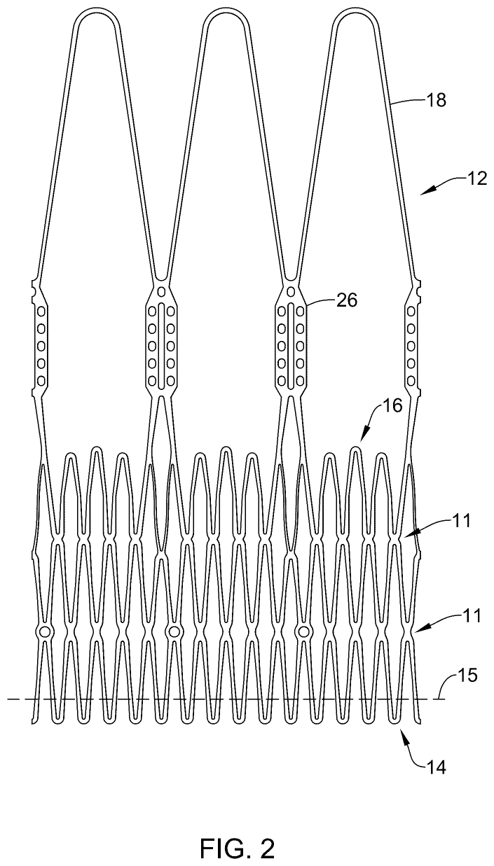

FIGS. 1-2 illustrate selected aspects of a replacement heart valve implant 10. It should be appreciated that the replacement heart valve implant 10 can be any type of replacement heart valve (e.g., a mitral valve, an aortic valve, etc.). Some non-limiting examples of the replacement heart valve implant 10 may include the ACURATE NEO2™, the ACURATE PRIME™, and/or family members thereof from Boston Scientific. Other examples are also contemplated. In use, the replacement heart valve implant 10 may be implanted (e.g., surgically or through transcatheter delivery) in a mammalian heart. The replacement heart valve implant 10 may be configured to allow one-way flow through the replacement heart valve implant 10 from an inflow end to an outflow end.

The replacement heart valve implant 10 may comprise an expandable framework 12. In some embodiments, the expandable framework 12 may have a substantially circular cross-section. In some embodiments, the expandable framework 12 may have a non-circular (e.g., D-shaped, elliptical, etc.) cross-section. In some embodiments, the expandable framework 12 may be formed from a resilient shape memory material. In at least some embodiments, the resilient shape memory material may be a metallic material such as nitinol. Some suitable but non-limiting examples of materials that may be used to form the expandable framework 12, including but not limited to metals and metal alloys, composites, polymers, and the like, are described below.

The replacement heart valve implant 10 and/or the expandable framework 12 may be configured to shift between a radially collapsed configuration and a radially expanded configuration. In some embodiments, the expandable framework 12 may be self-expanding. In some embodiments, the expandable framework 12 may be self-biased toward the radially expanded configuration. In some embodiments, the expandable framework 12 may be mechanically expandable. In some embodiments, the expandable framework 12 may be balloon expandable. Other configurations are also contemplated. In some embodiments, the expandable framework 12 may include and/or define a plurality of interstices (e.g., openings) through the expandable framework 12.

In some embodiments, the expandable framework 12 may comprise a tubular wall 13 including a plurality of interconnected struts and defining a central lumen extending along a central longitudinal axis of the expandable framework 12. In some embodiments, the tubular wall 13 may comprise and/or define an inflow end and an outflow end. The plurality of interconnected struts may comprise and/or define intersections 11 (e.g., FIG. 2) where interconnected struts of the plurality of interconnected struts join together (where the expandable framework 12 is monolithic and/or cut) and/or cross over and/or under each other (where the expandable framework 12 is braided, woven, etc.).

In some embodiments, the expandable framework 12 and/or the tubular wall 13 may include a plurality of lower crowns 14 extending upstream from upstreammost intersections 11 of the plurality of interconnected struts. In some embodiments, the plurality of lower crowns 14 may be disposed proximate and/or at the inflow end of the tubular wall 13. In some embodiments, the plurality of lower crowns 14 may comprise a bend line 15 (not shown in FIG. 1 and illustrated schematically with a dashed line in the flat pattern view of FIG. 2) extending circumferentially around the central longitudinal axis of the expandable framework 12 through each lower crown of the plurality of lower crowns 14. In some embodiments, the bend line 15 may be disposed upstream of the upstreammost intersections 11 of the plurality of interconnected struts and downstream of an upstreammost extent of the plurality of lower crowns 14. Additional details regarding the bend line 15 are disclosed below.

In some embodiments, the expandable framework 12 and/or the tubular wall 13 may include a plurality of upper crowns 16 extending downstream from downstreammost intersections 11 of the plurality of interconnected struts. In some embodiments, the plurality of upper crowns 16 may extend radially outward of and/or from the downstreammost intersections 11 of the plurality of interconnected struts in the radially expanded configuration. In some embodiments, the plurality of upper crowns 16 may be disposed proximate and/or at the outflow end of the tubular wall 13.

In some embodiments, the expandable framework 12 may include a plurality of stabilization arches 18 extending downstream from the outflow end, the tubular wall 13, and/or the plurality of upper crowns 16. In some embodiments, the plurality of stabilization arches 18 may extend downstream of and/or away from the plurality of upper crowns 16 in a direction opposite the plurality of lower crowns 14. In some embodiments, the plurality of upper crowns 16 may be disposed longitudinally and/or axially between the plurality of lower crowns 14 and the plurality of stabilization arches 18.

In some embodiments, the expandable framework 12 may comprise a plurality of commissure posts 26. In at least some embodiments, the plurality of commissure posts 26 may be disposed downstream of the outflow end and/or the plurality of upper crowns 16. In some embodiments, the plurality of commissure posts 26 may be disposed circumferentially around the central longitudinal axis. In some embodiments, the plurality of commissure posts 26 may be disposed longitudinally and/or axially between the plurality of upper crowns 16 and the plurality of stabilization arches 18. In some embodiments, the plurality of stabilization arches 18 may extend from the plurality of commissure posts 26.

In some embodiments, the replacement heart valve implant 10 may comprise a proximal portion and a distal portion. In some embodiments, orientation of the replacement heart valve implant 10 may be related to an implant delivery system and/or a direction of implantation relative to a target site (e.g., a native heart valve). In some embodiments, the proximal portion may comprise the outflow end and/or the plurality of stabilization arches 18. In some embodiments, the proximal portion may comprise the plurality of upper crowns 16 and/or the plurality of commissure posts 26. In some embodiments, the distal portion may comprise the inflow end and/or the plurality of lower crowns 14. Other configurations are also contemplated.

In some embodiments, the replacement heart valve implant 10 may comprise a plurality of valve leaflets 20 disposed within the central lumen. The plurality of valve leaflets 20 may be coupled, secured, and/or fixedly attached to the expandable framework 12 and/or the plurality of commissure posts 26. One or more means of securing the plurality of valve leaflets 20 to the expandable framework 12 and/or the plurality of commissure posts 26 may be used, including but not limited to, adhesive bonding, suturing, friction fit (e.g., pinching), etc. In some embodiments, the plurality of stabilization arches 18 may extend axially away from the plurality of valve leaflets 20 and/or from the plurality of commissure posts 26 or an attachment point (or attachment points) of the plurality of valve leaflets 20 with the expandable framework 12.

In some embodiments, each valve leaflet of the plurality of valve leaflets 20 may include a root edge coupled to the expandable framework 12 and a free edge (e.g., a coaptation edge) movable relative to the root edge to coapt with the free edges of the other leaflets along a coaptation region. In some embodiments, the plurality of valve leaflets 20 may be monolithically formed with each other, such that the plurality of valve leaflets 20 is formed as a single unitary and/or monolithic unit. In some embodiments, the plurality of valve leaflets 20 may be formed monolithically with other structures such as an inner skirt 22 and/or an outer skirt 24, base structures, liners, or the like.

The plurality of valve leaflets 20 may be configured to substantially restrict fluid from flowing through the replacement heart valve implant 10 and/or the central lumen in a closed position. For example, in some embodiments, the free edges of the plurality of valve leaflets 20 may move into coaptation with one another in the closed position to substantially restrict fluid from flowing through the replacement heart valve implant 10 and/or the central lumen. The free edges of the plurality of valve leaflets 20 may be spaced apart from each other in an open position to permit fluid flow through the replacement heart valve implant 10 and/or the central lumen. In FIG. 1, the plurality of valve leaflets 20 is shown in the open position or in a partially open position (e.g., a neutral position) that the plurality of valve leaflets 20 may move to when unbiased by fluid flow.

In some embodiments, the plurality of valve leaflets 20 may be comprised of a polymer, such as a thermoplastic polymer. In some embodiments, the plurality of valve leaflets 20 may include at least 50 percent by weight of a polymer. In some embodiments, the plurality of valve leaflets 20 may be formed from porcine pericardium, bovine pericardium, or other tissue. Other configurations and/or materials are also contemplated.

In some embodiments, the replacement heart valve implant 10 may include an inner skirt 22 disposed on and/or extending along an inner surface of the expandable framework 12 and/or the tubular wall 13. In at least some embodiments, the inner skirt 22 may be fixedly attached to the expandable framework 12 and/or the tubular wall 13. The inner skirt 22 may direct fluid, such as blood, flowing through the replacement heart valve implant 10 and/or the central lumen toward the plurality of valve leaflets 20. In some embodiments, the inner skirt 22 may be fixedly attached to and/or monolithically formed with the plurality of valve leaflets 20. The inner skirt 22 may ensure the fluid flows through the central lumen of the replacement heart valve implant 10 and does not flow around the plurality of valve leaflets 20 when they are in the closed position.

In some embodiments, the replacement heart valve implant 10 may include an outer skirt 24 disposed on and/or extending along the outer surface of the expandable framework 12 and/or the tubular wall 13. In some embodiments, the outer skirt 24 may be disposed at and/or adjacent the plurality of lower crowns 14. The outer skirt 24 may ensure the fluid flows through the central lumen of the replacement heart valve implant 10 and does not flow around the replacement heart valve implant 10 (e.g., between the expandable framework 12 and the vessel wall), so as to ensure that the plurality of valve leaflets 20 can stop the flow of fluid when in the closed position.

In some embodiments, the inner skirt 22 and/or the outer skirt 24 may include a polymer, and/or may include at least 50 percent by weight of a polymer. In some embodiments, the inner skirt 22 and/or the outer skirt 24 may be substantially impervious to fluid. In some embodiments, the inner skirt 22 and/or the outer skirt 24 may be formed from a thin tissue (e.g., porcine pericardium, bovine pericardium, or other tissue, etc.), a coated fabric material, or a nonporous and/or impermeable fabric material. Other configurations are also contemplated. Some suitable but non-limiting examples of materials that may be used to form the inner skirt 22 and/or the outer skirt 24 including but not limited to polymers, composites, and the like, are described below.

In some embodiments, the inner skirt 22 and/or the outer skirt 24 may seal one of, some of, or each of a plurality of interstices formed in the expandable framework 12 by and/or between the plurality of interconnected struts. In at least some embodiments, scaling one of, some of, or each of the plurality of interstices may be considered to prevent fluid from flowing through one of, some of, or each of the plurality of interstices of the expandable framework 12. In some embodiments, the inner skirt 22 and/or the outer skirt 24 may be attached to the expandable framework 12 using one or more methods including but not limited to tying with sutures or filaments, adhesive bonding, melt bonding, embedding or over molding, welding, etc.

In some embodiments, the expandable framework 12 and/or the replacement heart valve implant 10 may have an outer extent of about 23 millimeters (mm), about 25 mm, about 27 mm, about 30 mm, etc. in an unconstrained configuration (e.g., in the radially expanded configuration). In some embodiments, the expandable framework 12 and/or the replacement heart valve implant 10 may have an outer extent of about 10 mm, about 9 mm about 8 mm, about 7 mm, about 6 mm, etc. in the radially collapsed configuration. Other configurations are also contemplated.

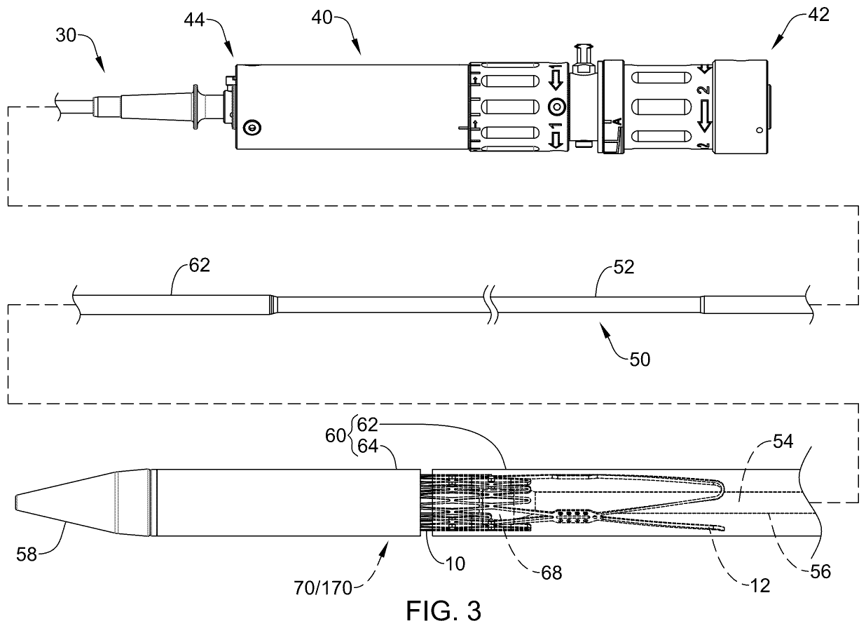

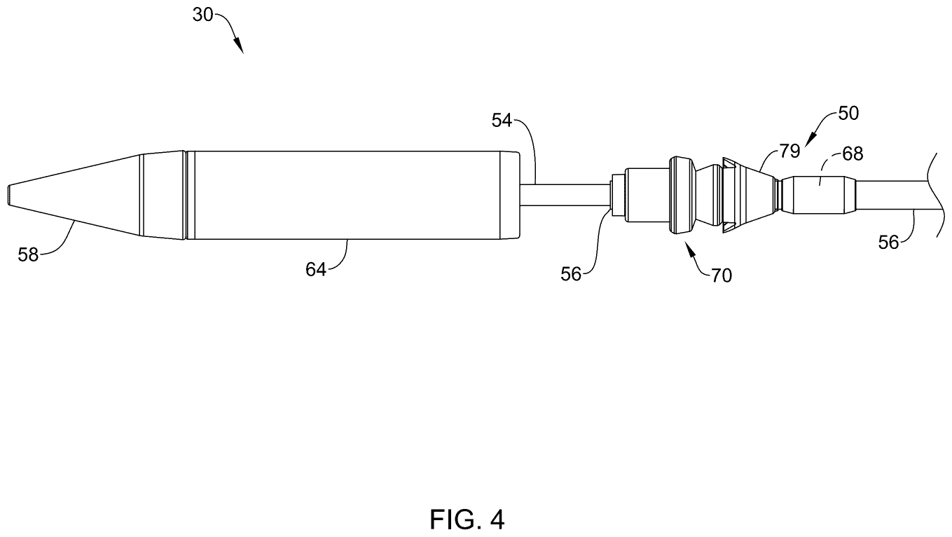

FIGS. 3-4 illustrate selected aspects of a replacement heart valve system including the replacement heart valve implant 10 and an implant delivery system 30 for delivering a replacement heart valve implant to a native heart valve (e.g., the aortic valve). The implant delivery system 30 may be compatible with and/or usable with the replacement heart valve implant 10. It should be noted that FIG. 3 includes at least one change of scale (e.g., all parts of the figure are not drawn to the same scale) to improve viewability and show additional detail of selected aspects of the implant delivery system 30. Additionally, the expandable framework 12 is shown in FIG. 3 in the radially collapsed configuration but some elements of the replacement heart valve implant 10 are not shown to improve clarity. In FIG. 4, the replacement heart valve implant 10 has been omitted.

The implant delivery system 30 may include a handle 40 and an elongate shaft assembly 50 extending distally from the handle 40. The handle 40 may include a first end 42 and a second end 44 opposite the first end 42. The elongate shaft assembly 50 may extend distally from the second end 44 of the handle 40. The handle 40 may include one or more rotatable knobs. In some embodiments, the one or more rotatable knobs may include a first rotatable knob and a second rotatable knob. In at least some embodiments, the first rotatable knob and/or the second rotatable knob may be configured to rotate around a central longitudinal axis of the implant delivery system 30 and/or the handle 40. Other configurations are also contemplated.

In some embodiments, a distal portion of the implant delivery system 30 and/or the elongate shaft assembly 50 may include an implant holding portion 60 configured to engage with and/or constrain the replacement heart valve implant 10 and/or the expandable framework 12 in the radially collapsed configuration. The elongate shaft assembly 50 may include an outer tubular member 52 extending distally from the handle 40 and an inner shaft 54 extending distally from the handle 40 within the outer tubular member 52 to a distal tip 58 disposed distal of the implant holding portion 60. In some embodiments, the implant holding portion 60 may comprise a proximal sheath 62 and a distal sheath 64. In some embodiments, the proximal sheath 62 and/or the distal sheath 64 may be formed from a polymeric material. In some embodiments, the proximal sheath 62 and/or the distal sheath 64 may include a reinforcing structure disposed therein and/or thereon. In some embodiments, the reinforcing structure may be a coil, a mesh, one or more filaments, bands, or strips, or another suitable structure. Other configurations are also contemplated.

In some embodiments, the inner shaft 54 may be slidably disposed within a lumen of the outer tubular member 52. In some embodiments, the elongate shaft assembly 50 may include an intermediate tubular member 56 disposed within and/or radially inward of the outer tubular member 52 and around and/or radially outward of the inner shaft 54. In at least some embodiments, the inner shaft 54 and the outer tubular member 52 are each axially translatable relative to the intermediate tubular member 56 independently of each other. For example, the inner shaft 54 may be translated relative to the intermediate tubular member 56 without translating the outer tubular member 52 relative to the intermediate tubular member 56, and vice versa.

In some embodiments, the proximal sheath 62 may be fixedly attached to the outer tubular member 52. In some embodiments, the proximal sheath 62 may be fixedly attached to and/or may extend distally from a distal end of the outer tubular member 52. In some embodiments, the distal sheath 64 and/or the distal tip 58 may be fixedly attached to the inner shaft 54. In some embodiments, the distal sheath 64 may be fixedly attached to the distal tip 58. In some embodiments, the distal sheath 64 may extend proximally from the distal tip 58. In some embodiments, the inner shaft 54 may include and/or at least partially define a guidewire lumen extending therethrough. In some embodiments, the guidewire lumen may extend through the handle 40.

In some embodiments, the handle 40 may be configured to manipulate and/or translate the proximal sheath 62 and/or the distal sheath 64 relative to each other using the first rotatable knob and/or the second rotatable knob. In some embodiments, the handle 40 may be configured to manipulate and/or translate the inner shaft 54 and/or the distal sheath 64 relative to the elongate shaft assembly 50, the outer tubular member 52, the intermediate tubular member 56, and/or the proximal sheath 62. In some embodiments, the handle 40 may be configured to manipulate and/or translate the outer tubular member 52 and/or the proximal sheath 62 relative to the elongate shaft assembly 50, the inner shaft 54, the intermediate tubular member 56, and/or the distal sheath 64.

During delivery of the replacement heart valve implant 10 to a treatment site (e.g., the native heart valve, the aortic valve, etc.), the replacement heart valve implant 10 may be disposed at least partially within the proximal sheath 62 and/or the distal sheath 64 in the radially collapsed configuration in a closed configuration of the implant holding portion 60. In some embodiments, the proximal sheath 62 and/or the distal sheath 64 may collectively define the implant holding portion 60 of the implant delivery system 30. In some embodiments, the implant holding portion 60 may be configured to constrain the replacement heart valve implant 10 in the radially collapsed configuration when the implant holding portion 60 is in the closed configuration. In some embodiments, the replacement heart valve implant 10 may be releasably coupled to and/or releasably engaged with the intermediate tubular member 56 and/or a stent holder 70 (or a stent holder 170, described below) when the replacement heart valve implant 10 is constrained within the implant holding portion 60 of the implant delivery system 30 in the radially collapsed configuration.

In some embodiments, the proximal sheath 62 may be configured to cover the proximal portion of the replacement heart valve implant 10 in the radially collapsed configuration when the implant holding portion 60 is in the closed configuration, and the distal sheath 64 may be configured to cover the distal portion of the replacement heart valve implant 10 in the radially collapsed configuration when the implant holding portion 60 is in the closed configuration. In some embodiments, the replacement heart valve implant 10 and/or the expandable framework 12 may be constrained in the radially collapsed configuration by the proximal sheath 62 and the distal sheath 64 in the closed configuration of the implant holding portion 60. In some embodiments, the proximal sheath 62 may be disposed adjacent to the distal sheath 64 in the closed configuration. In some embodiments, the proximal sheath 62 may abut the distal sheath 64 in the closed configuration. In some embodiments, the proximal sheath 62 may be axially spaced apart from the distal sheath 64 in the closed configuration. In some embodiments, the proximal sheath 62 may be axially spaced apart from the distal sheath 64 in the closed configuration by less than 20% of an overall length of the replacement heart valve implant 10 and/or the expandable framework 12. In some embodiments, the proximal sheath 62 may be axially spaced apart from the distal sheath 64 in the closed configuration by less than 15% of an overall length of the replacement heart valve implant 10 and/or the expandable framework 12. In some embodiments, the proximal sheath 62 may be axially spaced apart from the distal sheath 64 in the closed configuration by less than 10% of an overall length of the replacement heart valve implant 10 and/or the expandable framework 12. In some embodiments, the proximal sheath 62 may be axially spaced apart from the distal sheath 64 in the closed configuration by less than 5% of an overall length of the replacement heart valve implant 10 and/or the expandable framework 12. Other configurations are also contemplated.

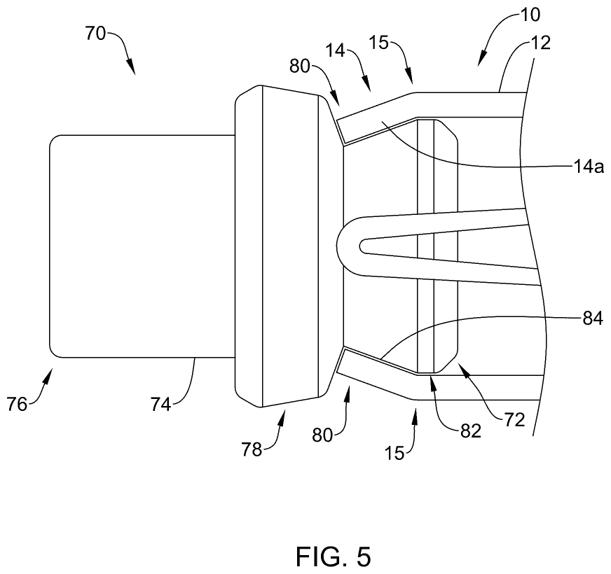

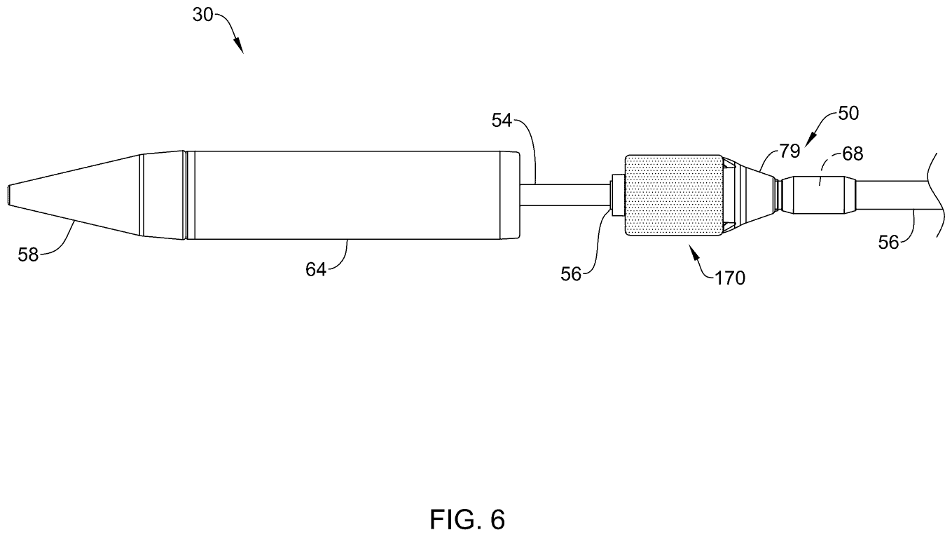

In some embodiments, the implant holding portion 60 and/or the elongate shaft assembly 50 may include the stent holder 70, which is shown in more detail in FIGS. 4-5, or the stent holder 170, which is shown in more detail in FIGS. 6-9. In at least some embodiments, the stent holder 70 and/or the stent holder 170 may be fixedly attached to the elongate shaft assembly 50. In some embodiments, the stent holder 70 and/or the stent holder 170 may be fixedly attached to the intermediate tubular member 56 of the elongate shaft assembly 50. In some embodiments, the stent holder 70 and/or the stent holder 170 may be integrally formed with the elongate shaft assembly 50 and/or the intermediate tubular member 56. In some embodiments, the stent holder 70 and/or the stent holder 170 may be configured to engage the expandable framework 12 in the radially collapsed configuration and/or when the replacement heart valve implant 10 is constrained within the implant holding portion 60 of the implant delivery system 30.

The implant delivery system 30 and/or the elongate shaft assembly 50 may include a primary visual indicator 68 configured and/or adapted to be visible under fluoroscopy with an imaging device. Other imaging means suitable for use with transcatheter surgical procedures are also contemplated. The implant delivery system 30 and/or the primary visual indicator 68 may be configured to cooperate with the imaging device to position the replacement heart valve implant 10 at a desired insertion depth within the native heart valve (e.g., the aortic valve).

In use, the implant delivery system 30 may be advanced percutaneously through the vasculature to a position adjacent to the treatment site (e.g., the native valve annulus). For example, the implant delivery system 30 may be advanced through the vasculature and across the aortic arch to a position adjacent to the native heart valve (e.g., the aortic valve). Alternative approaches to treat a defective aortic valve and/or other heart valve(s) are also contemplated with the implant delivery system 30.

The desired insertion depth may be selected to maximize radially outward force of the expandable framework 12 within the native heart valve (e.g., the aortic valve). Positioning the replacement heart valve implant 10 at the desired insertion depth and/or within a maximum tolerance from the desired insertion depth, the replacement heart valve implant 10 and/or the expandable framework 12 may exhibit optimal arching within the native heart valve (e.g., the aortic valve) and thereby prevent migration of the replacement heart valve implant 10 and/or the expandable framework 12 downstream (or upstream).

Positioning the replacement heart valve implant 10 and/or the expandable framework 12 within the native heart valve (e.g., the aortic valve) may be accomplished by locating the primary visual indicator 68 relative to the native heart valve (e.g., the aortic valve). During visualization, the native heart valve (e.g., the aortic valve) may be identified and/or visualized under fluoroscopy using known means and/or methods, such as contrast injection.

FIG. 4 illustrates selected aspects of the implant delivery system 30 of FIG. 3 in accordance with the disclosure. For improved clarity, the replacement heart valve implant 10 and some portions of the implant delivery system 30 are not shown. For example, in FIG. 4, the distal sheath 64 is shown while the proximal sheath 62 is not shown.

In some embodiments, the primary visual indicator 68 may be fixedly attached to the elongate shaft assembly 50 and/or the intermediate tubular member 56. In some embodiments, the primary visual indicator 68 may be a marker band embedded within the elongate shaft assembly 50. In some embodiments, the primary visual indicator 68 may be fixedly attached to the elongate shaft assembly 50 and/or the intermediate tubular member 56 by a shrink wrap or by an adhesive element. Other configurations are also contemplated. As discussed herein, the primary visual indicator 68 may be configured and/or adapted to be visible under fluoroscopy with the imaging device. The primary visual indicator 68 may be formed from a radiopaque material. Some suitable but non-limiting examples of radiopaque materials for the primary visual indicator 68 are described below.

In some embodiments, the implant delivery system 30 and/or the elongate shaft assembly 50 may include the stent holder 70 configured to engage the expandable framework 12 of the replacement heart valve implant 10 in the radially collapsed configuration and/or when the replacement heart valve implant 10 is constrained within the implant holding portion 60 of the implant delivery system 30. Some aspects related to the stent holder 70 are shown in more detail in FIG. 5. As shown in FIG. 5, the stent holder 70 may include a body 74, a first end portion 72 extending proximally from the body 74, and a second end portion 76 disposed opposite the first end portion 72.

In some embodiments, the stent holder 70 may have a groove 80 extending circumferentially in an outer surface of the stent holder 70. In some embodiments, the stent holder 70 may comprise a medial shoulder 78 extending radially outward from the body 74 at and/or adjacent a medial portion of the stent holder 70. In some embodiments, the stent holder 70 may comprise a proximal shoulder 82 adjacent the first end portion 72. In at least some embodiments, the groove 80 may be disposed proximal of the medial shoulder 78. In some embodiments, the groove 80 may be disposed distal of the proximal shoulder 82 and/or the first end portion 72. In some embodiments, the groove 80 may be disposed between the first end portion 72 and the medial shoulder 78. In some embodiments, the groove 80 may be disposed between the proximal shoulder 82 and the medial shoulder 78.

The groove 80 may be configured to receive the plurality of lower crowns 14 in the radially collapsed configuration, as seen in FIG. 5. In some embodiments, the groove 80 may be configured to receive every lower crown of the plurality of lower crowns 14 in the radially collapsed configuration. For improved clarity, a limited number of lower crowns are illustrated in FIG. 5, and it shall be understood that there may be more lower crowns present than those expressly illustrated. In some embodiments, the groove 80 may be configured to receive the plurality of lower crowns 14 and/or every lower crown of the plurality of lower crowns 14 in any rotational orientation and/or all rotational orientations of the expandable framework 12 and/or the replacement heart valve implant 10 relative to the implant delivery system 30 and/or the stent holder 70. In some embodiments, the replacement heart valve system may have no predetermined and/or required relative rotational orientation of the expandable framework 12 and/or the replacement heart valve implant 10 with respect to the implant delivery system 30 and/or the stent holder 70 in order for the groove 80 to receive the plurality of lower crowns 14 and/or every lower crown of the plurality of lower crowns 14. For example, there may be no structure on the expandable framework 12 that must rotationally align with a corresponding structure on the stent holder 70 in order for the groove 80 to receive the plurality of lower crowns 14 and/or every lower crown of the plurality of lower crowns 14 (e.g., no peg that must engage an aperture, etc.)

In some embodiments, the bend line 15 may define a radially outermost extent of the plurality of lower crowns 14. In some embodiments, the bend line 15 may define the radially outermost extent of the plurality of lower crowns 14 in the radially collapsed configuration. In some embodiments, the bend line 15 may define the radially outermost extent of the plurality of lower crowns 14 in the radially expanded configuration. In some embodiments, the upstreammost extent of the plurality of lower crowns 14 may be disposed radially inward of the bend line 15 and/or the radially outermost extent of the plurality of lower crowns 14 in the radially collapsed configuration and/or in the radially expanded configuration. In some embodiments, the upstreammost extent of the plurality of lower crowns 14 may still be disposed radially inward of the bend line 15 after the replacement heart valve implant 10 and/or the expandable framework 12 has been shifted to the radially expanded configuration.

In some embodiments, an upstream portion 14A of the plurality of lower crowns 14 may extend from the bend line 15 upstream to the upstreammost extent of the plurality of lower crowns 14. In some embodiments, the upstream portion 14A and/or the upstreammost extent of the plurality of lower crowns 14 may extend into the groove 80 in the radially collapsed configuration. In some embodiments, the proximal shoulder 82 may be configured to engage the plurality of lower crowns 14 at the bend line 15 in the radially collapsed configuration.

In some embodiments, the upstream portion 14A of the plurality of lower crowns 14 may be oriented at an acute angle to the central longitudinal axis of the expandable framework 12, the central longitudinal axis of the elongate shaft assembly 50, and/or a central longitudinal axis of the stent holder 70. In some embodiments, the acute angle to the central longitudinal axis of the expandable framework 12, the central longitudinal axis of the elongate shaft assembly 50, and/or the central longitudinal axis of the stent holder 70 may be between about zero degrees and about 90 degrees (e.g., greater than about zero degrees and less than about 90 degrees). In some embodiments, the acute angle to the central longitudinal axis of the expandable framework 12, the central longitudinal axis of the elongate shaft assembly 50, and/or the central longitudinal axis of the stent holder 70 may be between about zero degrees and about 60 degrees (e.g., greater than about zero degrees and less than about 60 degrees). In some embodiments, the acute angle to the central longitudinal axis of the expandable framework 12, the central longitudinal axis of the elongate shaft assembly 50, and/or the central longitudinal axis of the stent holder 70 may be between about zero degrees and about 45 degrees (e.g., greater than about zero degrees and less than about 45 degrees). In some embodiments, the acute angle to the central longitudinal axis of the expandable framework 12, the central longitudinal axis of the elongate shaft assembly 50, and/or the central longitudinal axis of the stent holder 70 may be between about 15 degrees and about 30 degrees (e.g., greater than or equal to about 15 degrees and less than or equal to about 30 degrees). Other configurations and/or angles between zero and 90 degrees are also contemplated. In some alternative embodiments, the upstream portion 14A of the plurality of lower crowns 14 may be oriented at a right angle and/or at 90 degrees to the central longitudinal axis of the expandable framework 12, the central longitudinal axis of the elongate shaft assembly 50, and/or a central longitudinal axis of the stent holder 70.

In some embodiments, the groove 80 may comprise an angled surface 84 configured to matingly engage with the upstream portion 14A of the plurality of lower crowns 14 extending from the bend line 15 upstream to the upstreammost extent of the plurality of lower crowns 14 in the radially collapsed configuration. In some embodiments, the upstream portion 14A of the plurality of lower crowns 14 may be configured to abut the angled surface 84 of the groove 80 in the radially collapsed configuration.

In some embodiments, the angled surface 84 of the groove 80 may be oriented at the acute angle to the central longitudinal axis of the expandable framework 12, the central longitudinal axis of the elongate shaft assembly 50, and/or the central longitudinal axis of the stent holder 70 of the upstream portion 14A of the plurality of lower crowns 14 discussed above. In some alternative configurations, the angled surface 84 of the groove 80 may be oriented at a different angle to the central longitudinal axis of the expandable framework 12, the central longitudinal axis of the elongate shaft assembly 50, and/or the central longitudinal axis of the stent holder 70 than the upstream portion 14A of the plurality of lower crowns 14 discussed above.

In some embodiments, the stent holder 70 may be configured and/or adapted to be visible under fluoroscopy. In some embodiments, the stent holder 70 may be formed from stainless steel or another radiopaque material. Some suitable but non-limiting materials for the stent holder 70 are described below.

In some embodiments, the first end portion 72, the proximal shoulder 82, the groove 80, and/or the angled surface 84 may be configured and/or adapted to engage the plurality of lower crowns 14 of the expandable framework 12 of the replacement heart valve implant 10 (not shown) in the radially collapsed configuration and/or when the replacement heart valve implant 10 is constrained within the implant holding portion 60 of the implant delivery system 30 such that proximal axial movement of the replacement heart valve implant 10 and/or the expandable framework 12 relative to the stent holder 70 and/or the elongate shaft assembly 50 is prevented. In some embodiments, a proximal portion and/or a proximal facing surface of the medial shoulder 78 may be configured and/or adapted to engage the plurality of lower crowns 14 of the expandable framework 12 of the replacement heart valve implant 10 in the radially collapsed configuration and/or when the replacement heart valve implant 10 is constrained within the implant holding portion 60 of the implant delivery system 30 such that distal axial movement of the replacement heart valve implant 10 and/or the expandable framework 12 relative to the stent holder 70 and/or the elongate shaft assembly 50 is prevented.

In some embodiments, the distal sheath 64 may extend proximally from the distal tip 58, as seen in FIG. 4. In some embodiments, the implant delivery system 30 and/or the elongate shaft assembly 50 may include a cage (not shown) disposed radially outward of and/or extending radially outward from the elongate shaft assembly 50 and/or the second end portion 76 of the stent holder 70. In some embodiments, the cage may be positioned radially outward of and axially overlapping the body 74 of the stent holder 70. In some embodiments, the cage may be configured to substantially center the distal sheath 64 over and/or around the elongate shaft assembly 50 and/or the replacement heart valve implant 10 (not shown) as the distal sheath 64 is moved from an open configuration to the closed configuration. Other configurations are also contemplated.

In some embodiments, the implant delivery system 30 and/or the implant holding portion 60 may include an atraumatic transition shield 79, as seen in FIG. 4. The atraumatic transition shield 79 may be disposed adjacent the stent holder 70. In some embodiments, the atraumatic transition shield 79 may be disposed between the stent holder 70 and the handle 40. In some embodiments, the atraumatic transition shield 79 may be disposed proximal the stent holder 70. In some embodiments, the atraumatic transition shield 79 may be disposed at and/or adjacent the first end portion 72 of the stent holder 70. In some embodiments, the atraumatic transition shield 79 may axially overlap the first end portion 72 of the stent holder 70. In some embodiments, the atraumatic transition shield 79 may be disposed radially outward of at least a portion of the first end portion 72 of the stent holder 70. In some embodiments, the atraumatic transition shield 79 may be tapered radially inward in the proximal direction and/or toward the handle 40. The atraumatic transition shield 79 may be configured to prevent the replacement heart valve implant 10, the expandable framework 12, the plurality of valve leaflets 20, etc. (not shown in FIG. 4) from catching on the stent holder 70 as the implant delivery system 30 is withdrawn after deploying the replacement heart valve implant 10.

In use, after advancing and/or navigating the implant delivery system 30 and/or the implant holding portion 60 to the treatment site (over a guidewire, for example), the proximal sheath 62 and/or the distal sheath 64 may be axially translated relative to each other to shift the implant holding portion 60 to the open configuration. When unconstrained by the implant holding portion 60, the replacement heart valve implant 10 (e.g., FIG. 1) and/or the expandable framework 12 (e.g., FIG. 1) may be configured to shift from the radially collapsed configuration (e.g., FIG. 3) to the radially expanded configuration (e.g., FIG. 1) by manipulating the one or more rotatable knobs on the handle 40. Shifting the replacement heart valve implant 10 (e.g., FIG. 1) and/or the expandable framework 12 toward the radially expanded configuration after axially translating the proximal sheath 62 and/or the distal sheath 64 away from each other may permit the replacement heart valve implant 10 (e.g., FIG. 1) and/or the expandable framework 12 to decouple and/or detach from the implant delivery system 30. Some suitable but non-limiting materials for the implant delivery system 30, the handle 40, the elongate shaft assembly 50, and/or components or elements thereof, for example metallic materials and/or polymeric materials, are described below.

FIG. 6 illustrates selected aspects of the implant delivery system 30 of FIG. 3 in accordance with the disclosure. For improved clarity, the replacement heart valve implant 10 and some portions of the implant delivery system 30 are not shown. For example, in FIG. 6, the distal sheath 64 is shown while the proximal sheath 62 is not shown. Additionally, aspects that are unchanged from the discussion above with respect to FIG. 4, see discussion related to the primary visual indicator 68 for example, are not repeated in the interest of brevity.

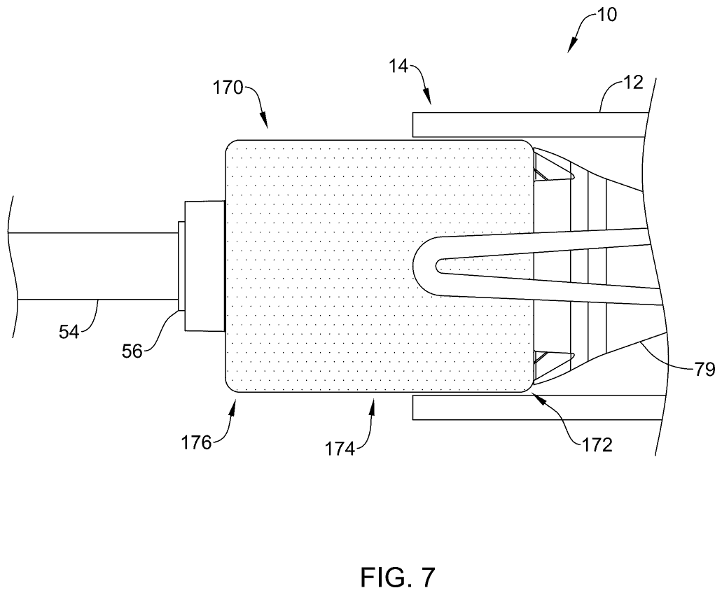

In some embodiments, the implant delivery system 30 and/or the elongate shaft assembly 50 may include the stent holder 170 configured to engage the expandable framework 12 of the replacement heart valve implant 10 in the radially collapsed configuration and/or when the replacement heart valve implant 10 is constrained within the implant holding portion 60 of the implant delivery system 30. Some aspects related to the stent holder 170 are shown in more detail in FIGS. 7-9. As shown in FIG. 7, the stent holder 170 may include a body 174, a first end portion 172 extending proximally from the body 174, and a second end portion 176 disposed opposite the first end portion 172. In FIG. 7, the expandable framework 12 is shown with the plurality of lower crowns 14 disposed over the first end portion 172 of the stent holder 170 in a partially collapsed configuration. This is indicative of the replacement heart valve implant 10 being partially loaded into the implant holding portion 60 of the implant delivery system 30.

In some embodiments, the stent holder 170 may be formed from a conformable material. In some embodiments, the stent holder 170 may be formed from a dense cell foam. In some embodiments, the stent holder 170 and/or portions thereof (e.g., the first end portion 172, etc.) may be configured to be radially compressed as the replacement heart valve implant 10 and/or the expandable framework 12 is engaged therewith and/or shifted toward the radially collapsed configuration. In some embodiments, one or more physical properties of the conformable material may be altered, changed, enhanced, etc. as the stent holder 170 is radially compressed. For example, radial compression of the stent holder 170 may cause the conformable material to become more rigid. In some embodiments, one or more physical properties of the conformable material may be altered, changed, enhanced, etc. as the stent holder 170 changes temperature and/or as the stent holder 170 is exposed to changes in temperature. For example, a change in temperature of the stent holder 170 may cause the conformable material to become more malleable or more rigid. In one non-limiting concept, the stent holder 170 may be malleable at a first temperature (e.g., less than body temperature) as the replacement heart valve implant 10 and/or the expandable framework 12 is engaged therewith and/or shifted toward the radially collapsed configuration, and the stent holder 170 may be less malleable and/or more rigid at a second temperature higher than the first temperature (e.g., body temperature). Other configurations, including configurations opposite those explicitly described and/or configurations that combine various properties, are also contemplated.

In some embodiments, the stent holder 170 may be configured and/or adapted to be visible under fluoroscopy. In some embodiments, the stent holder 170 may include a radiopaque material. In some embodiments, the conformable material may include a radiopaque material disposed therein and/or may comprise a radiopaque mixture. Other configurations are also contemplated. Some suitable but non-limiting materials for the stent holder 170 are described below.

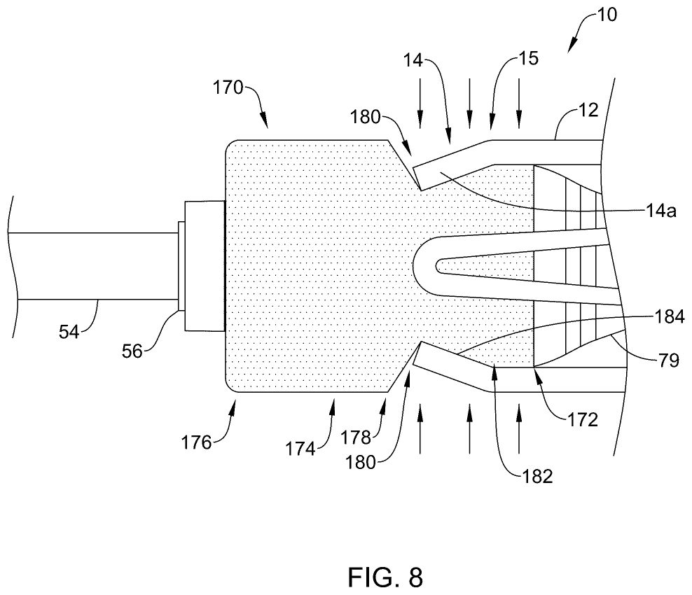

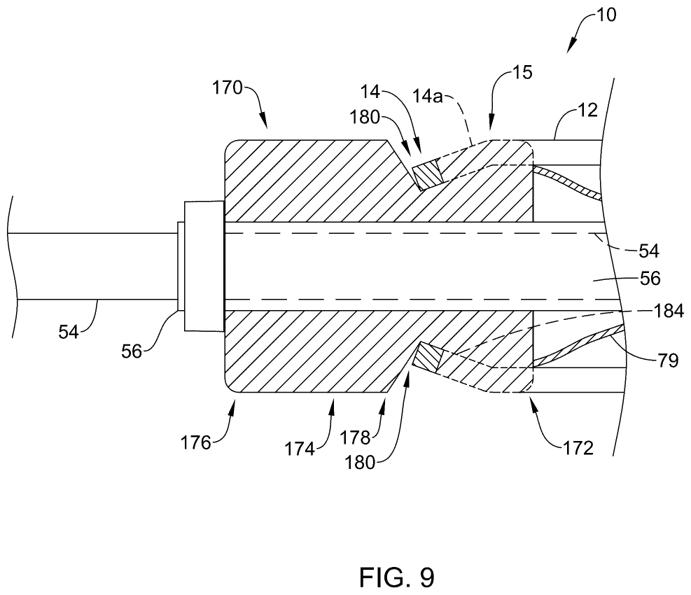

In some embodiments, the stent holder 170 may have a groove 180 extending circumferentially in an outer surface of the stent holder 170, as seen in FIGS. 8-9. In at least some embodiments, the groove 180 may be formed by and/or during radially collapsing (e.g., crimping) the expandable framework 12 and/or the plurality of lower crowns 14 onto the stent holder 170. For example, a tool or die may be used to radially collapse and/or crimp that expandable framework 12 and/or the plurality of lower crowns 14 toward and/or to the radially collapsed configuration. The tool or die may comprise features that simultaneously form the groove 180 in the outer surface of the stent holder 170. In some alternative configurations, the groove 180 may be formed in the outer surface of the stent holder 170 prior to radially collapsing (e.g., crimping) the expandable framework 12 and/or the plurality of lower crowns 14 onto the stent holder 170, and/or toward and/or to the radially collapsed configuration. Other configurations are also contemplated.

In some embodiments, the conformable material may be configured to extend between the plurality of lower crowns 14 in the radially collapsed configuration such that the upstream portion 14A of the plurality of lower crowns 14 is substantially surrounded by the stent holder 170 and/or the conformable material on two sides (e.g., on a radially inward facing side and on a proximal facing side). In some embodiments, the conformable material may be configured to extend between the plurality of lower crowns 14 in the radially collapsed configuration such that the upstream portion 14A of the plurality of lower crowns 14 is substantially surrounded by the stent holder 170 and/or the conformable material on three sides (e.g., on all by a radially outward facing side).

In some embodiments, the stent holder 170 may comprise a medial shoulder 178 extending circumferentially in the body 174 at and/or adjacent a medial portion of the stent holder 170. In some embodiments, the stent holder 170 may comprise a proximal shoulder 182 adjacent the first end portion 172. In some embodiments, the medial shoulder 178 and the proximal shoulder 182 may at least partially define the groove 180. In at least some embodiments, the groove 180 may be disposed proximal of the medial shoulder 178. In some embodiments, the groove 180 may be disposed distal of the proximal shoulder 182 and/or the first end portion 172. In some embodiments, the groove 180 may be disposed between the first end portion 172 and the medial shoulder 178. In some embodiments, the groove 180 may be disposed between the proximal shoulder 182 and the medial shoulder 178.

The groove 180 may be configured to receive the plurality of lower crowns 14 in the radially collapsed configuration, as seen in FIGS. 8-9. In some embodiments, the groove 180 may be configured to receive every lower crown of the plurality of lower crowns 14 in the radially collapsed configuration. For improved clarity, a limited number of lower crowns are illustrated in FIGS. 8-9, and it shall be understood that there may be more lower crowns present than those expressly illustrated.

In some embodiments, the bend line 15 may define a radially outermost extent of the plurality of lower crowns 14. In some embodiments, the bend line 15 may define the radially outermost extent of the plurality of lower crowns 14 in the radially collapsed configuration. In some embodiments, the bend line 15 may define the radially outermost extent of the plurality of lower crowns 14 in the radially expanded configuration. In some embodiments, the upstreammost extent of the plurality of lower crowns 14 may be disposed radially inward of the bend line 15 and/or the radially outermost extent of the plurality of lower crowns 14 in the radially collapsed configuration and/or in the radially expanded configuration. In some embodiments, the upstreammost extent of the plurality of lower crowns 14 may still be disposed radially inward of the bend line 15 after the replacement heart valve implant 10 and/or the expandable framework 12 has been shifted to the radially expanded configuration.

In some embodiments, an upstream portion 14A of the plurality of lower crowns 14 may extend from the bend line 15 upstream to the upstreammost extent of the plurality of lower crowns 14. In some embodiments, the upstream portion 14A and/or the upstreammost extent of the plurality of lower crowns 14 may extend into the groove 180 in the radially collapsed configuration. In some embodiments, the proximal shoulder 182 may be configured to engage the plurality of lower crowns 14 at the bend line 15 in the radially collapsed configuration. In some embodiments, the upstream portion 14A of the plurality of lower crowns 14 may be bent radially inward at the bend line 15 by and/or during radially collapsing (e.g., crimping) the expandable framework 12 and/or the plurality of lower crowns 14 onto the stent holder 170. In some alternative configurations, the upstream portion 14A of the plurality of lower crowns 14 may be bent radially inward at the bend line 15 prior to radially collapsing (e.g., crimping) the expandable framework 12 and/or the plurality of lower crowns 14 onto the stent holder 170, and/or toward and/or to the radially collapsed configuration. Other configurations are also contemplated.

In some embodiments, the upstream portion 14A of the plurality of lower crowns 14 may be oriented at an acute angle to the central longitudinal axis of the expandable framework 12, the central longitudinal axis of the elongate shaft assembly 50, and/or a central longitudinal axis of the stent holder 170. In some embodiments, the acute angle to the central longitudinal axis of the expandable framework 12, the central longitudinal axis of the elongate shaft assembly 50, and/or the central longitudinal axis of the stent holder 170 may be between about zero degrees and about 45 degrees (e.g., greater than about zero degrees and less than about 45 degrees). In some embodiments, the acute angle to the central longitudinal axis of the expandable framework 12, the central longitudinal axis of the elongate shaft assembly 50, and/or the central longitudinal axis of the stent holder 70 may be between about 15 degrees and about 30 degrees (e.g., greater than or equal to about 15 degrees and less than or equal to about 30 degrees). Other configurations and/or angles between zero and 45 degrees are also contemplated.

In some embodiments, the groove 180 may comprise an angled surface 184 configured to matingly engage with the upstream portion 14A of the plurality of lower crowns 14 extending from the bend line 15 upstream to the upstreammost extent of the plurality of lower crowns 14 in the radially collapsed configuration. In some embodiments, the upstream portion 14A of the plurality of lower crowns 14 may be configured to abut the angled surface 184 of the groove 180 in the radially collapsed configuration.

In some embodiments, the angled surface 184 of the groove 180 may be oriented at the acute angle to the central longitudinal axis of the expandable framework 12, the central longitudinal axis of the elongate shaft assembly 50, and/or the central longitudinal axis of the stent holder 170 of the upstream portion 14A of the plurality of lower crowns 14 discussed above. In some alternative configurations, the angled surface 184 of the groove 180 may be oriented at a different angle to the central longitudinal axis of the expandable framework 12, the central longitudinal axis of the elongate shaft assembly 50, and/or the central longitudinal axis of the stent holder 170 than the upstream portion 14A of the plurality of lower crowns 14 discussed above.

In some embodiments, the first end portion 172, the proximal shoulder 182, the groove 180, and/or the angled surface 184 may be configured and/or adapted to engage the plurality of lower crowns 14 of the expandable framework 12 of the replacement heart valve implant 10 (not shown) in the radially collapsed configuration and/or when the replacement heart valve implant 10 is constrained within the implant holding portion 60 of the implant delivery system 30 such that proximal axial movement of the replacement heart valve implant 10 and/or the expandable framework 12 relative to the stent holder 170 and/or the elongate shaft assembly 50 is prevented. In some embodiments, a proximal portion and/or a proximal facing surface of the medial shoulder 178 may be configured and/or adapted to engage the plurality of lower crowns 14 of the expandable framework 12 of the replacement heart valve implant 10 in the radially collapsed configuration and/or when the replacement heart valve implant 10 is constrained within the implant holding portion 60 of the implant delivery system 30 such that distal axial movement of the replacement heart valve implant 10 and/or the expandable framework 12 relative to the stent holder 170 and/or the elongate shaft assembly 50 is prevented.

In some embodiments, the distal sheath 64 may extend proximally from the distal tip 58, as seen in FIG. 6. In some embodiments, the implant delivery system 30 and/or the elongate shaft assembly 50 may include a cage (not shown) disposed radially outward of and/or extending radially outward from the elongate shaft assembly 50 and/or the second end portion 176 of the stent holder 170. In some embodiments, the cage may be positioned radially outward of and axially overlapping the body 174 of the stent holder 170. In some embodiments, the cage may be configured to substantially center the distal sheath 64 over and/or around the elongate shaft assembly 50 and/or the replacement heart valve implant 10 (not shown) as the distal sheath 64 is moved from an open configuration to the closed configuration. Other configurations are also contemplated.

In some embodiments, the implant delivery system 30 and/or the implant holding portion 60 may include an atraumatic transition shield 79, as seen in FIG. 6. The atraumatic transition shield 79 may be disposed adjacent the stent holder 170. In some embodiments, the atraumatic transition shield 79 may be disposed between the stent holder 170 and the handle 40. In some embodiments, the atraumatic transition shield 79 may be disposed proximal the stent holder 170. In some embodiments, the atraumatic transition shield 79 may be disposed at and/or adjacent the first end portion 172 of the stent holder 170. In some embodiments, the atraumatic transition shield 79 may axially overlap the first end portion 172 of the stent holder 170. In some embodiments, the atraumatic transition shield 79 may be disposed radially outward of at least a portion of the first end portion 172 of the stent holder 170. In some embodiments, the atraumatic transition shield 79 may be tapered radially inward in the proximal direction and/or toward the handle 40. The atraumatic transition shield 79 may be configured to prevent the replacement heart valve implant 10, the expandable framework 12, the plurality of valve leaflets 20, etc. (not shown in FIG. 6) from catching on the stent holder 170 as the implant delivery system 30 is withdrawn after deploying the replacement heart valve implant 10.

In use, after advancing and/or navigating the implant delivery system 30 and/or the implant holding portion 60 to the treatment site (over a guidewire, for example), the proximal sheath 62 and/or the distal sheath 64 may be axially translated relative to each other to shift the implant holding portion 60 to the open configuration. When unconstrained by the implant holding portion 60, the replacement heart valve implant 10 (e.g., FIG. 1) and/or the expandable framework 12 (e.g., FIG. 1) may be configured to shift from the radially collapsed configuration (e.g., FIG. 3) to the radially expanded configuration (e.g., FIG. 1) by manipulating the one or more rotatable knobs on the handle 40. Shifting the replacement heart valve implant 10 (e.g., FIG. 1) and/or the expandable framework 12 toward the radially expanded configuration after axially translating the proximal sheath 62 and/or the distal sheath 64 away from each other may permit the replacement heart valve implant 10 (e.g., FIG. 1) and/or the expandable framework 12 to decouple and/or detach from the implant delivery system 30. Some suitable but non-limiting materials for the implant delivery system 30, the handle 40, the elongate shaft assembly 50, and/or components or elements thereof, for example metallic materials and/or polymeric materials, are described below.

In some embodiments, the upstream portion 14A may extend radially inward from the bend line 15 and/or the radially outermost extent of the plurality of lower crowns 14, and/or the upstreammost extent of the plurality of lower crowns 14 may be disposed radially inward of the bend line 15 and/or the radially outermost extent of the plurality of lower crowns 14, after the replacement heart valve implant 10 and/or the expandable framework 12 has shifted to the radially expanded configuration. In some embodiments, this may prevent the upstreammost extent of the plurality of lower crowns 14 from traumatically engaging with and/or injuring adjacent tissue after implantation within the native heart valve. In some alternative embodiments, the upstream portion 14A and/or the upstreammost extent of the plurality of lower crowns 14 may be configured to shift radially outward as the replacement heart valve implant 10 and/or the expandable framework 12 shifts toward and/or to the radially expanded configuration. In some embodiments, this may permit the plurality of lower crowns 14 to further engage with the native heart valve and/or the native valve annulus to increase resistance to migration of the replacement heart valve implant 10. In such embodiments, care should be taken and/or design considerations must be made to prevent the plurality of lower crowns 14 from traumatically engaging with and/or injuring adjacent tissue after implantation. For example, atraumatic features may be included and/or added to the plurality of lower crowns 14. Other configurations are also contemplated.

In some embodiments, a method of loading a replacement heart valve implant 10 into the replacement heart valve system and/or an implant delivery system 30 may comprise positioning a replacement heart valve implant 10, as described herein, adjacent an implant holding portion 60 of an implant delivery system 30, as discussed and shown herein.

In some embodiments, the method of loading the replacement heart valve implant 10 into the replacement heart valve system and/or the implant delivery system 30 may comprise positioning the plurality of lower crowns 14 surrounding the stent holder 70 or the stent holder 170 of the implant delivery system 30.

In some embodiments, the method of loading the replacement heart valve implant 10 into the replacement heart valve system and/or the implant delivery system 30 may comprise radially collapsing the replacement heart valve implant 10 and/or the expandable framework 12 to the radially collapsed configuration such that the plurality of lower crowns 14 extends into the groove 80 or the groove 180 extending circumferentially in the outer surface of the stent holder 70 or the stent holder 170, respectively. In some embodiments, the method of loading the replacement heart valve implant 10 into the replacement heart valve system and/or the implant delivery system 30 may comprise radially collapsing the replacement heart valve implant 10 and/or the expandable framework 12 to the radially collapsed configuration such that every lower crown of the plurality of lower crowns 14 extends into the groove 80 or the groove 180 extending circumferentially in the outer surface of the stent holder 70 or the stent holder 170, respectively. In at least some embodiments, radially collapsing the replacement heart valve implant 10 and/or the expandable framework 12 to the radially collapsed configuration may be done according to known methods and/or processes, and/or using known means, tools, equipment, etc.

In at least some embodiments, the upstream portion 14A may extend radially inward from the bend line 15 and/or the radially outermost extent of the plurality of lower crowns 14, and/or the upstreammost extent of the plurality of lower crowns 14 may be disposed radially inward of the bend line 15 and/or the radially outermost extent of the plurality of lower crowns 14, before and/or prior to radially collapsing the replacement heart valve implant 10 and/or the expandable framework 12 to the radially collapsed configuration. Other configurations are also contemplated.

In some embodiments, radially collapsing the replacement heart valve implant 10 and/or the expandable framework 12 to the radially collapsed configuration may bend the upstream portion 14A of the plurality of lower crowns 14 radially inward at the bend line 15. In some embodiments, radially collapsing the replacement heart valve implant 10 and/or the expandable framework 12 to the radially collapsed configuration may form the groove 180 in the outer surface of the stent holder 170. Other configurations are also contemplated.

In some embodiments, the method of loading the replacement heart valve implant 10 into the replacement heart valve system and/or the implant delivery system 30 may comprise shifting the implant holding portion 60 of the implant delivery system 30 over the replacement heart valve implant 10 to a closed configuration. In some embodiments, shifting the implant holding portion 60 of the implant delivery system 30 over the replacement heart valve implant 10 to the closed configuration may constrain the replacement heart valve implant 10 in the radially collapsed configuration.

In some embodiments, shifting the distal sheath 64 of the implant holding portion 60 toward and/or to the closed configuration may constrain and/or retain the plurality of lower crowns 14 and/or the inflow end within the groove 80 (or the groove 180) such that axial movement of the replacement heart valve implant 10 and/or the expandable framework 12 relative to the implant delivery system 30 and/or the stent holder 70 (or the stent holder 170) is substantially prevented.

In at least some interventions, the replacement heart valve implant 10 (e.g., FIG. 1) may be deployed within the native heart valve (e.g., the native heart valve is left in place). Alternatively, the replacement heart valve implant 10 (e.g., FIG. 1) may be deployed within a previously replaced native heart valve (such as via a surgical aortic valve replacement procedure or via a transcatheter aortic valve replacement procedure) and the replacement heart valve implant 10 may be deployed within the earlier-placed implant.

The materials that can be used for the various components of the replacement heart valve system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the replacement heart valve implant, the expandable framework, the implant delivery system, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-clastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten and alloys thereof, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine);
antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A replacement heart valve implant, comprising:
an expandable framework configured to shift between a radially collapsed configuration and a radially expanded configuration; and
a plurality of valve leaflets secured to the expandable framework;
wherein the expandable framework comprises a tubular wall including a plurality of interconnected struts, a plurality of upper crowns extending downstream from downstreammost intersections of the plurality of interconnected struts, and a plurality of lower crowns extending upstream from upstreammost intersections of the plurality of interconnected struts;
wherein the plurality of lower crowns comprises a bend line extending circumferentially around a central longitudinal axis of the expandable framework through each lower crown, the bend line being disposed upstream of the upstreammost intersections of the plurality of interconnected struts and downstream of an upstreammost extent of the plurality of lower crowns.

2. The replacement heart valve implant of claim 1, wherein the bend line defines a radially outermost extent of the plurality of lower crowns.

3. The replacement heart valve implant of claim 2, wherein the upstreammost extent of the plurality of lower crowns is disposed radially inward of the radially outermost extent of the plurality of lower crowns.

4. The replacement heart valve implant of claim 1, wherein an upstream portion of the plurality of lower crowns extending from the bend line upstream to the upstreammost extent of the plurality of lower crowns is oriented at an acute angle to the central longitudinal axis of the expandable framework.

5. The replacement heart valve implant of claim 4, wherein the acute angle is greater than zero degrees and less than 45 degrees.

6. The replacement heart valve implant of claim 5, wherein the acute angle is greater than 15 degrees and less than 30 degrees.

7. A replacement heart valve system, comprising:
a replacement heart valve implant, comprising:
an expandable framework configured to shift between a radially collapsed configuration and a radially expanded configuration; and
a plurality of valve leaflets secured to the expandable framework;
wherein the expandable framework comprises a tubular wall including a plurality of interconnected struts, a plurality of upper crowns extending downstream from downstreammost intersections of the plurality of interconnected struts, and a plurality of lower crowns extending upstream from upstreammost intersections of the plurality of interconnected struts;
wherein the plurality of lower crowns comprises a bend line extending circumferentially around a central longitudinal axis of the expandable framework through each lower crown, the bend line being disposed upstream of the upstreammost intersections of the plurality of interconnected struts and downstream of an upstreammost extent of the plurality of lower crowns; and
an implant delivery system, comprising:
a handle; and
an elongate shaft assembly extending distally from the handle, wherein a distal portion of the elongate shaft assembly includes an implant holding portion configured to engage with the replacement heart valve implant in the radially collapsed configuration.

8. The replacement heart valve system of claim 7, wherein the implant holding portion comprises:
a proximal sheath configured to cover a proximal portion of the replacement heart valve implant in the radially collapsed configuration; and
a distal sheath configured to cover a distal portion of the replacement heart valve implant in the radially collapsed configuration.

9. The replacement heart valve system of claim 7, wherein the implant holding portion comprises a stent holder having a groove extending circumferentially in an outer surface of the stent holder, the groove being configured to receive the plurality of lower crowns in the radially collapsed configuration.

10. The replacement heart valve system of claim 9, wherein the groove is configured to receive every lower crown of the plurality of lower crowns in the radially collapsed configuration.

11. The replacement heart valve system of claim 9, wherein the groove comprises an angled surface configured to matingly engage with an upstream portion of the plurality of lower crowns extending from the bend line upstream to the upstreammost extent of the plurality of lower crowns.

12. The replacement heart valve system of claim 9, wherein the groove comprises a first shoulder configured to engage the plurality of lower crowns at the bend line.

13. The replacement heart valve system of claim 9, wherein the stent holder is formed from a conformable material configured to extend between the plurality of lower crowns in the radially collapsed configuration.

14. The replacement heart valve system of claim 13, wherein the groove is formed by crimping the plurality of lower crowns onto the stent holder.

15. A method of loading a replacement heart valve implant into a replacement heart valve system, comprising:
positioning a replacement heart valve implant adjacent an implant holding portion of an implant delivery system, the replacement heart valve implant comprising:
an expandable framework configured to shift between a radially collapsed configuration and a radially expanded configuration; and
a plurality of valve leaflets secured to the expandable framework;
wherein the expandable framework comprises a tubular wall including a plurality of interconnected struts, a plurality of upper crowns extending downstream from downstreammost intersections of the plurality of interconnected struts, and a plurality of lower crowns extending upstream from upstreammost intersections of the plurality of interconnected struts;
wherein the plurality of lower crowns comprises a bend line extending circumferentially around a central longitudinal axis of the expandable framework through each lower crown, the bend line being disposed upstream of the upstreammost intersections of the plurality of interconnected struts and downstream of an upstreammost extent of the plurality of lower crowns;
positioning the plurality of lower crowns surrounding a stent holder of the implant delivery system;
radially collapsing the expandable framework to the radially collapsed configuration such that the plurality of lower crowns extends into a groove extending circumferentially in an outer surface of the stent holder; and
shifting the implant holding portion of the implant delivery system over the replacement heart valve implant to a closed configuration.

16. The method of claim 15, wherein an upstream portion of the plurality of lower crowns extending from the bend line upstream to the upstreammost extent of the plurality of lower crowns is oriented at an acute angle to the central longitudinal axis of the expandable framework.

17. The method of claim 16, wherein the upstream portion of the plurality of lower crowns abuts an angled surface of the groove in the radially collapsed configuration.

18. The method of claim 16, wherein the upstream portion of the plurality of lower crowns extends radially inward from the bend line prior to radially collapsing the expandable framework.

19. The method of claim 15, wherein radially collapsing the expandable framework to the radially collapsed configuration bends the plurality of lower crowns radially inward at the bend line.

20. The method of claim 19, wherein radially collapsing the expandable framework to the radially collapsed configuration forms the groove in the outer surface of the stent holder.
